(19) United States
(12) Patent Application Publication
(10) Pub. No.: US 2025/0383349 A1
(43) Pub. Date: Dec. 18, 2025

(54) METHODS AND DEVICES FOR QUANTITATIVELY ESTIMATING THROMBOMODULIN

(71) Applicant: DioTeX Diagnostics, LLC, Baltimore, MD (US)

(72) Inventors: Cassandra PARENT, Baltimore, MD (US); Eric SIMON, Baltimore, MD (US); Feiyang HUANG, Baltimore, MD (US); Amanda RUCI, Baltimore, MD (US); Ellie ZHANG, Baltimore, MD (US)

(73) Assignee: DioTeX Diagnostics, LLC, Baltimore, MD (US)

(21) Appl. No.: 19/240,058

(22) Filed: Jun. 17, 2025

Related U.S. Application Data
(60) Provisional application No. 63/661,103, filed on Jun. 18, 2024.

(51) Int. Cl. G01N 33/543; B82Y 15/00; G01N 33/68

(52) U.S. Cl. CPC G01N 33/54388; G01N 33/54346; G01N 33/6893; B82Y 15/00; G01N 2333/7452; G01N 2800/22; G01N 2800/52

(57) ABSTRACT

This disclosure provides methods and devices for determining a quantitative estimate of thrombomodulin levels in a mammalian subject suspected of internal hemorrhaging. The method includes applying a blood sample from the subject to a handheld assay device capable of providing optical quantitation of the amount of thrombomodulin in the sample, measuring, by means of said assay device, an analyte signal value correlated to a concentration of the thrombomodulin in the blood sample and comparing the analyte signal value to a minimum threshold, wherein an analyte signal value less than the minimum threshold indicates that the subject is not internally hemorrhaging, and an analyte signal value above the minimum threshold indicates the subject is internally hemorrhaging. The methods and devices are adapted to rapidly assess internal hemorrhaging and hemorrhagic shock in a patient outside of hospital settings.

### CROSS-REFERENCE TO RELATED APPLICATIONS

This application claims benefit of priority from U.S. Provisional Patent Application No. 63/661,103, filed Jun. 18, 2024, the entire contents of which are incorporated herein by reference.

### SEQUENCE LISTING

The instant application contains an electronic sequence listing. The contents of the electronic sequence listing 6507.001A Sequence Listing XML.xml; Size: 6,372 bytes; and Date of Creation: Jun. 5, 2025, is herein incorporated by reference in its entirety.

### TECHNICAL FIELD

This disclosure relates to methods and devices for quantitatively estimating thrombomodulin levels in a mammalian subject. Advantageously, the methods and devices are adapted to rapidly assess internal hemorrhaging and hemorrhagic shock in a patient outside of hospital settings.

### BACKGROUND

Trauma-induced hemorrhage is the leading cause of death for Americans under 46 and causes up to 90% of preventable U.S. troop deaths. Heavy internal or external bleeding as a result of trauma can cause hemorrhagic shock, a condition where the body shuts off critical functions such as breathing and consciousness. Early diagnosis is critical for administering emergency treatment in the field or rapid evacuation to a surgical facility that can administer proper treatment. While external bleeding is easy to visually detect, internal bleeding generally requires a physician's suspicion and subsequent examination.

Within hospital settings, these examinations can be confirmed via imaging and the patient can easily receive the prompt surgical care necessary for survival. However, outside of hospital, imaging is impractical for point-of-injury hemorrhage detection and the standard of care relies on standard hemodynamic measurements, which are inadequate indicators of internal bleeding up until failure of the body's compensatory mechanisms. Subsequent delays in internal hemorrhage diagnosis lead to larger blood loss and increased resuscitation requirements, making early detection of internal hemorrhage critical for patient survival.

Current methods of point-of-injury diagnostics of internal hemorrhage remain inadequate, and there remains a need for a fast, accessible, and accurate tool to minimize treatment delays and loss of life.

### SUMMARY

We provide methods and devices for determining a quantitative estimate of thrombomodulin levels in a mammalian subject suspected of internal hemorrhaging. The method includes applying a blood sample from the subject to a handheld assay device capable of providing optical quantification of the amount of thrombomodulin in the sample, measuring, by means of said assay device, an analyte signal value correlated to a concentration of the thrombomodulin in the blood sample and comparing the analyte signal value to a minimum threshold, wherein an analyte signal value less than the minimum threshold indicates that the subject is not internally hemorrhaging, and an analyte signal value above the minimum threshold indicates the subject is internally hemorrhaging. The methods and devices are adapted to rapidly assess internal hemorrhaging and hemorrhagic shock in a patient outside of hospital settings.

### BRIEF DESCRIPTION OF THE FIGURES

FIG. 1 shows an exemplary device of this disclosure.

FIG. 2 shows an exploded view of an exemplary device of this disclosure.

### DETAILED DESCRIPTION

This disclosure provides methods and devices for quantitatively estimating thrombomodulin levels in a mammalian subject to assess internal hemorrhaging and hemorrhagic shock. The methods and devices of this disclosure advantageously improve existing technology by providing a form factor that is both highly portable and fast-acting. The devices can be easily carried by field medics or EMTs and used in the field to rapidly confirm if a patient is likely to be suffering from internal hemorrhaging and hemorrhagic shock. The ability to obtain confirmation of internal hemorrhaging and hemorrhagic shock in the field, especially in the early stages, means that a patient can be identified for treatment on site and evacuated to a hospital during a critical window after onset of injury. This is especially useful in triage situations where many people may have been injured and field medics must identify those most critically injured and in need of immediate care. Early determination and treatment of internal hemorrhaging and hemorrhagic can vastly improve patient outcomes.

Aspects of this disclosure utilize the chemical mechanism of a thrombomodulin anti-thrombomodulin antibody reaction. Thrombomodulin is an endothelial cell surface transmembrane protein encoded by the THBD gene (also referred to as THBD and CD141). The amino acid sequence is provided in SEQ ID: 1. The extracellular domain of thrombomodulin is provided in SEQ ID NO: 2. The transmembrane domain is provided in SEQ ID NO: 3. The cytosolic domain is provided in SEQ ID NO: 4.

Thrombomodulin is found on the surface of endothelial cells and plays an important anticoagulation role. Thrombomodulin is a thrombin receptor and is a cofactor for thrombin-mediated activation of protein C, which is important in maintaining the homeostasis of thrombosis. Immediately after blood vessel injury the glycocalyx, the lining of blood vessels, is shed from endothelial cells due to shear stress from altered blood flow. Thrombomodulin is one of the components of the glycocalyx. Patients who undergo traumatic injury experience elevated levels of thrombomodulin after injury. Normal healthy adults have approximately 3.1±1.3 ng/ml. of thrombomodulin in their blood, and patients experiencing hemorrhage have an average of 6.91±2.08 ng/mL of thrombomodulin in their blood. In addition, mice models demonstrated that this increase in concentration occurs early in the earliest stage of hemorrhage, meaning that there is still time for effective treatment to occur. As an early-stage biomarker of internal hemorrhaging and hemorrhagic shock, thrombomodulin has been identified by the inventors as a useful analyte in a biology based test to assess and diagnose internal bleeding.

Aspects of this disclosure relate to methods and devices taking advantage of the biochemical and optical principles of a “one-pot” assay and applying them in an accessible form factor. For example, preferred devices and methods may employ a lateral flow assay (LFA) in order to simplify sample preparation and advantageously may provide colorimetric test results that can be interpreted without laboratory equipment. Preferred examples utilize blood microsampling, such as finger prick method, in order to collect a test specimen. This specimen will then be transferred to the collection site on the device. Within 5 to 15 minutes, the presence of a color shifted line on the device will indicate an elevated level of thrombomodulin correlated with internal hemorrhaging. The absence of such a line will indicate a lack of internal hemorrhaging in the patient.

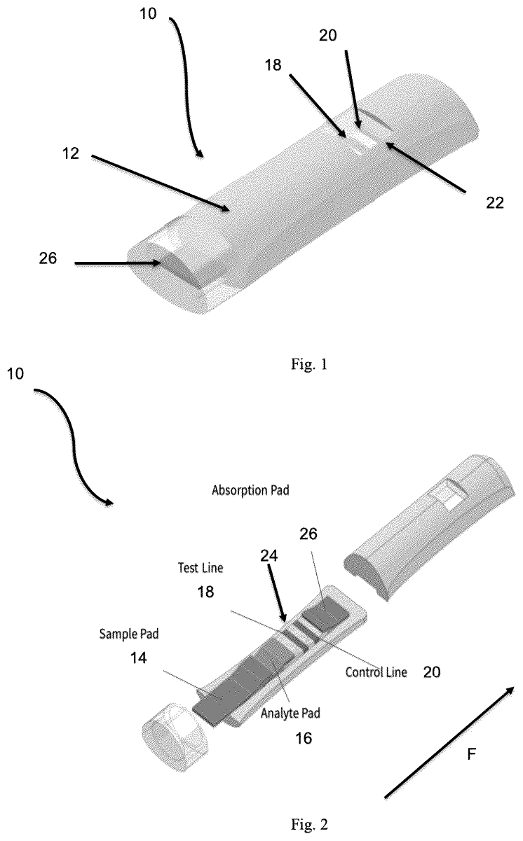

FIGS. 1 and 2 depict an exemplary assay device 10 for quantitative estimation of thrombomodulin levels in a mammalian (e.g., human) subject configured as a lateral flow assay (LFA) device. The assay device 10 is constructed with housing 12 containing a sample pad 14, a single release pad 16 and at least one capture region 18, in this order. The sample pad 14, signal release pad 16 and capture region 18 are fluidly connected to provide a flowpath for the sample and an analyte contained therein to move from the sample pad 14 to the capture region 16. Generally, an absorption pad 26 may also be provided to draw the fluid through the assay device 10 by capillary action and absorb any excess fluid. The direction of the flowpath is indicated by “F” in FIG. 2.

The sample pad 14 is positioned at a first end of the housing 12 and is accessible from the housing 12 so as to allow the sample pad 14 to receive a sample, such as a drop of blood. A sample received by the sample pad 14 is typically conducted by capillary action into the housing 12 and along the flowpath.

The signal release pad 16 contains a signal member with binding specificity for thrombomodulin impregnated therein. The signal member is freely diffusible and able to be carried by fluid flow from the signal release pad 16 to the capture region 18. The signal member preferably produces an optically detectable analyte signal when the signal member is captured in the capture region 18 in an amount exceeding a minimum threshold.

The minimum threshold for producing the analyte signal may be selected but those skilled in the art to correlate with a concentration of thrombomodulin in the blood sample indicative of internal hemorrhaging or hemorrhagic shock. In this way, an analyte signal value less than the minimum threshold may indicate that the subject is not internally hemorrhaging or in hemorrhagic shock, and an analyte signal value above the minimum threshold may indicate the subject is internally hemorrhaging or in hemorrhagic shock. In preferred examples, the minimum threshold correlates to a concentration of thrombomodulin in the blood sample of at least 3.1 ng/mL. However, the minimum threshold may correlate to a concentration of thrombomodulin in the blood sample of at least 3.4 ng/ml, at least 4.4 ng/mL, at least 5.4 ng/ml, at least 6.4 ng/ml, at least 7.4 ng/ml, at least 8.4 ng/ml, or at least 9 ng/ml.

The signal member may be any suitable material that produces a signal that is optically detectable. Examples of an optically detectable signal may be a colorimetric change or fluorescence. An optically detectable signal may be observable by the naked eye or by using a device.

In some examples, the signal member may comprise a conjugated nanoparticle having an anti-thrombomodulin antibody immobilized thereon, Nanoparticles may be made of fold, fold-coated silicon, latex, or other suitable materials. Gold nanoparticles are preferred. The size of the particles and particle size distribution is preferably selected to allow the particles to be carried by fluid flow from the signal release pad 16 to the capture region 18. A suitable particle size may be 50 to 120 nm.

The capture region 18 is fluidly connected to the signal release pad 16 and has binding specificity for thrombomodulin may be provided by immobilizing anti-thrombomodulin antibodies to a substrate 24 in the capture region 18. The substrate 24 may be any suitable material, such as a porous membrane, nitrocellulose membrane, and the like.

Anti-thrombomodulin antibodies provided as part of the signal member (primary antibodies) and in the capture region 18 (secondary antibodies) are typically selected for binding different regions of the thrombomodulin protein so that both antibodies may bind simultaneously. Anti-thrombomodulin antibodies may be those commercially available or may be prepared by methods known in the art. Suitable commercially available antibodies include Thrombomodulin Polyclonal Antibody manufactured by Proteintech (14318-1-AP) which binds to the sequence identified in SEQ ID NO:5.

The assay device 10 may also include a control region 20, preferably positioned after the capture region 18 in terms of the flow path. The control region 20 has binding specificity for thrombomodulin antibodies and produces an optically detectable control signal when the thrombomodulin antibodies are captured by the control region 20. In this way, the control region 20 enables a user to verify that the sample fluid has successfully flowed through the assay device and verify a negative reading.

Binding specificity for thrombomodulin antibodies may be provided by immobilizing anti-antibodies specific for the selected thrombomodulin antibody of the signal member to the substrate 24 in the control region 20. An anti-antibody specific for the thrombomodulin antibodies is not limited so long as it has binding specificity for the selected thrombomodulin antibody. A suitable anti-antibody specific for the thrombomodulin antibody may be, for example, an anti-species antibody, such as a goat anti-mouse antibody.

The assay device 10 may include more than one capture region 18 fluidly connected to the signal release pad. For example, multiple capture regions may be provided by immobilizing additional lines of reagent material along the flowpath in a ladder arrangement. Alternatively, the device may include multiple parallel flowpaths with one or more respective capture regions.

Advantageously, each of the plurality of capture regions 18 may have different sensitivities for different concentrations of thrombomodulin in the blood samples. For example, the capture regions 18 may have different respective binding affinities for thrombomodulin or different concentrations of anti-thrombomodulin antibodies. The use of a plurality of capture regions 18 having different respective binding sensitivities for an analyte can be used to provide a quantitative determination of the amount of analyte in the sample. The design of a quantitative lateral flow assay is known in the art. “See, e.g., U.S. Pat. No. 9,851,364, incorporated herein in its entirety by reference.

An assay device having plurality of capture regions 18 may include a first analyte threshold corresponding to a first concentration of thrombomodulin and a second (or further) analyte threshold corresponding to a second (or further) concentration of thrombomodulin. Thus, for an analyte signal value of less than the second threshold, but greater than the first threshold, a concentration of thrombomodulin elevated relative to normal levels in the blood sample, for example, may be indicated. An analyte signal value of greater than the second threshold may be indicated as a further elevated concentration of thrombomodulin in the blood sample. According to an example, the assay device may comprise first and second analyte thresholds which correspond respectively to a minimum threshold concentration of thrombomodulin in the blood sample of at least 3.1 ng/mL and to at least 5.6 ng/mL. The result may indicate a concentration range of thrombomodulin in the blood sample of a patient to help the user to confirm the likelihood of internal hemorrhaging and assess its severity.

Preferably, the housing 12 is configured as a hand-held device. Hand-held construction is advantageous for transporting and using the device in the field, such as a battlefield. The housing 12 may also be provided with a window 22 positioned over the capture region 18 to allow a user to visually inspect the capture region 18.

LFA devices are generally known in the art. See, e.g., U.S. Pat. No. 7,144,742, incorporated herein in its entirety by reference. Materials constituting the flow path F may be selected by those skilled in art. For example, the flowpath may be provided by one or more substrates that are able to convey a liquid from a first position to a second position and may be for example a capillary channel, a microfluidic pathway, or a porous carrier such as a lateral flow porous carrier material which may overlap in a linear or stacked arrangements or which are fluidically connected. The porous carrier materials may be the same or different. For example, untreated paper, cellulose blends, nitrocellulose, polyester, an acrylonitrile copolymer, rayon, glass fiber, and the like may also be employed as support matrix materials to provide flow. Suitably, the material of the signal release pad may be either glass fiber or cellulose filter. Buffers may also be used as known in the art. A suitable buffer may be 5 mM Borate, 10% sucrose, and 5% trehalose.

Methods of using the assay device 10 will now be described. A subject may be evaluated for trauma and, if the subject is suspected of suffering from internal hemorrhaging, a user (e.g., field medic) will perform an internal hemorrhage assay. Generally, the user will obtain a blood sample from a mammalian subject suspected of internal hemorrhaging, such as by finger prick. The blood sample may be applied to the sample pad 14 on a sample receiving portion 26 of the assay device 10. Capillary flow may pull the sample liquid through substrate 24 into the signal release pad 16 containing signal members which, in this example, are anti-thrombomodulin conjugated gold nanoparticles (GNPs). If thrombomodulin is present in the blood sample, it will bind to single members and form a thrombomodulin-GNP complex. The thrombomodulin-GNP complex will be pulled further through the assay device 10 into substrate 24 and onto the capture region 18 containing immobilized anti-thrombomodulin antibodies, thereby immobilizing the complex in the capture region 18. This action will result in a “sandwich ELISA” in which the target thrombomodulin protein is caught between two antibodies (the primary and the secondary). The sample liquid flows further to the control region 20, which binds to any thrombomodulin-GNP complexed GNPs. Any remaining liquid will continue along the flow path until it reaches the absorption pad 26 at the end of the assay device, which will absorb the liquid.

Binding of the thrombomodulin-GNP complexes in the capture region produces an optically detectable analyte signal value that may indicate if thrombomodulin is present and in an amount exceeding the minimum threshold, thereby indicating whether the patient is internally hemorrhaging. A positive test result may have two clearly visible lines corresponding to the capture region and control region. A negative test result may have only one clearly visible line (the control line).

In this example, if the analyte signal value is greater than or equal to a minimum threshold correlated to a concentration of the thrombomodulin in the blood sample of at least 3.1 ng/mL, the subject may be considered to be experiencing internal hemorrhaging. If the assay is positive, the patient may immediately be signaled for medevac. Additionally, the subject may be immediately administered a blood product transfusion, such as plasma. As the assay device may provide a result within 5 minutes, treatment may begin immediately. For example, immediate treatment may generally occur in the field prior to transporting the subject to a hospital setting.

### Sequences

### UniProt Consortium. (2024). Thrombomodulin (Entry P07204).

ORGANISM: Homo sapiens
SEQ ID NO 1
APAEPQPGGSQCVEHDCFALYPGPATFLNASQICDGLRGHLMTVR
SSVAADVISLLINGDGGVGRRRLWIGLQLPPGCGDPKRLGPLRGF
QWVTGDNNTSYSRWARLDLNGAPLCGPLCVAVSAAEATVPSEPIW
EEQQCEVKADGFLCEFHFPATCRPLAVEPGAAAAAVSITYGTPFA
ARGADFQALPVGSSAAVAPLGLQLMCTAPPGAVQGHWAREAPGAW
DCSVENGGCEHACNAIPGAPRCQCPAGAALQADGRSCTASATQSC
NDLCEHFCVPNPDQPGSYSCMCETGYRLAADQHRCEDVDDCILEP
SPCPQRCVNTQGGFECHCYPNYDLVDGECVEPVDPCFRANCEYQC
QPLNQTSYLCVCAEGFAPIPHEPHRCQMFCNQTACPADCDPNTQA
SCECPEGYILDDGFICTDIDECENGGFCSGVCHNLPGTFECICGP
DSALARHIGTDCDSGKVDGGDSGSGEPPPSPTPGSTLTPPAVGLV
HSGLLIGISIASLCLVVALLALLCHLRKKQGAARAKMEYKCAAPS
KEVVLQHVRTERTPQRL
LENGTH: 497
TYPE: PRT
ORGANISM: Homo sapiens
SEQ ID NO 2
APAEPQPGGSQCVEHDCFALYPGPATFLNASQICDGLRGHLMTVR
SSVAADVISLLLNGDGGVGRRRLWIGLQLPPGCGDPKRLGPLRGF
QWVTGDNNTSYSRWARLDLNGAPLCGPLCVAVSAAEATVPSEPIW
EEQQCEVKADGFLCEFHFPATCRPLAVEPGAAAAAVSITYGTPFA
ARGADFQALPVGSSAAVAPLGLQLMCTAPPGAVQGHWAREAPGAW
DCSVENGGCEHACNAIPGAPRCQCPAGAALQADGRSCTASATQSC
NDLCEHFCVPNPDQPGSYSCMCETGYRLAADQHRCEDVDDCILEP
SPCPQRCVNTQGGFECHCYPNYDLVDGECVEPVDPCFRANCEYQC
QPLNQTSYLCVCAEGFAPIPHEPHRCQMFCNQTACPADCDPNTQA
SCECPEGYILDDGFICTDIDECENGGFCSGVCHNLPGTFECICGP
DSALARHIGTDCDSGKVDGGDSGSGEPPPSPTPGSTLTPPAVGLV
HS
LENGTH: 4
TYPE: PRT
ORGANISM: Homo sapiens
SEQ ID NO 3
GLLIGISIASLCLVVALLALLCHL
LENGTH: 36
TYPE: PRT
ORGANISM: Homo sapiens
SEQ ID NO 4
RKKQGAARAKMEYKCAAPSKEVVLQHVRTERTPQRL
LENGTH: 333
TYPE: PRT
ORGANISM: Homo sapiens
SEQ ID NO 5
PAPAEPQPGGSQCVEHDCFALYPGPATFLNASQICDGLRGHLMTV
RSSVAADVISLLLNGDGGVGRRRLWIGLQLPPGCGDPKRLGPLRG
FQWVTGDNNTSYSRWARLDLNGAPLCGPLCVAVSAAEATVPSEPI
WEEQQCEVKADGFLCEFHFPATCRPLAVEPGAAAAAVSITYGTPF
AARGADFQALPVGSSAAVAPLGLQLMCTAPPGAVQGHWAREAPGA
WDCSVENGGCEHACNAIPGAPRCQCPAGAALQADGRSCTASATQS
CNDLCEHFCVPNPDQPGSYSCMCETGYRLAADQHRCEDVDDCILE
PSPCPQRCVNTQGGFECH

What is claimed is:

1. A method for determining a quantitative estimate of thrombomodulin levels in a mammalian subject suspected of internal hemorrhaging, the method comprising:
a) applying a blood sample from the subject to a hand-held assay device capable of providing optical quantitation of the amount of thrombomodulin in the sample;
b) measuring, by means of said assay device, an analyte signal value correlated to a concentration of the thrombomodulin in the blood sample;
c) comparing the analyte signal value to a minimum threshold, wherein an analyte signal value less than the minimum threshold indicates that the subject is not internally hemorrhaging, and an analyte signal value above the minimum threshold indicates the subject is internally hemorrhaging.

2. The method according to claim 1, wherein the minimum threshold correlates to a concentration of the thrombomodulin in the blood sample of more than 3.1 ng/ml within the extracted blood serum.

3. The method according to claim 1 wherein the minimum threshold correlates to a concentration of thrombomodulin in the blood of more than 5.6 ng/ml within the extracted blood serum.

4. The method according to claim one wherein a time between applying step and the comparing step is between 10 seconds and 5 minutes.

5. The method, according to claim, further comprising verifying a determination of the quantitative estimate of thrombomodulin levels in the mammalian subject by comparing the analyte signal to a second minimum threshold correlated to a concentration of the thrombomodulin of the thrombomodulin in the blood sample of at least 5.6 ng/ml.

6. The method according to claim 1, further compromising verifying a determination of the quantitative estimate of thrombomodulin levels in the mammalian subject by comparing the analyte signal value to a control signal.

7. The method according to claim 1, wherein the assay device comprises:
a. a housing configured as a hand-held device;
b. a sample pad for receiving a blood sample
c. a signal release pad fluidly connected to the sample pad and containing a signal member with binding specificity for thrombomodulin, and
d. a capture region fluidly connected to the signal release pad and in which a capture reagent having binding specificity for thrombomodulin
e. wherein the signal member produces an optically detectable analyte signal when an amount of the signal member captured by the capture region correlates to a concentration of the thrombomodulin in the blood sample exceeding the minimum threshold.

8. An assay or assay device for quantitative estimation of thrombomodulin levels comprising:
a. a housing configured as a hand-held device;
b. a sample pad for receiving a blood sample;
c. a signal release pad fluidly connected to the sample pad and containing a signal member with binding specificity for thrombomodulin, and
d. a capture region fluidly connected to the signal release pad and in which a capture reagent having binding specificity for thrombomodulin
e. wherein the signal member produces an optically detectable analyte signal when an amount of the signal member captured by the capture region correlates to a concentration of the thrombomodulin in the blood sample exceeding the minimum threshold.

9. The assay device of claim 8, wherein the minimum threshold correlates to a concentration of the thrombomodulin in the blood sample of at least 3.1 ng/ml of the blood sample.

10. The assay device of claim 8, wherein the signal member is a nanoparticle.

11. The assay device of claim 8, wherein the signal member is a gold nanoparticle having an average particle diameter of 50 nm to 120 nm.

12. The assay device of claim 8, further comprising a second capture region fluidly connected to the signal release pad in which a second capture reagent having binding specificity for thrombomodulin is immobilized.

13. The assay device of claim 8, further comprising a control region that binds the signal member wherein the control region produces an optically detectable control signal when the signal member is captured by the control region.

14. The assay device of claim 8, wherein the housing comprises a window and the capture region is positioned in the window.

15. The assay device of claim 8, wherein the assay device produces the optically detectable analyte signal within 10 minutes after a sample containing thrombomodulin in a concentration exceeding the minimum threshold is applied to the sample pad.

16. A method of treating a mammalian subject suspected of internal hemorrhaging comprising:
a. applying a blood sample from the subject to a hand-held assay device capable of providing optical quantification of the amount of thrombomodulin in the sample.
b. measuring, my means of said assay or assay device, an analyte signal value correlated to a concentration of the thrombomodulin in the blood sample.
c. comparing the analyte signal value to a minimum threshold correlated to a concentration of the thrombomodulin in the blood sample, wherein an analyte signal value less than the minimum threshold indicated that the subject is not internally hemorrhaging, and an analyte signal value above the minimum threshold indicates the subject is internally hemorrhaging; and
d. administering a blood product transfusion to the subject if the analyte signal value is above the minimum threshold.
e. Administering a blood product transfusion to the subject if the analyte signal value is above the minimum threshold

17. The method of treating a mammalian subject according to claim 16, wherein the minimum threshold is correlated to a concentration of the thrombomodulin in the blood sample of at least 3.1 ng/ml

18. The method of treating a mammalian subject according to claim 16, wherein the minimum threshold is correlated to a concentration of thrombomodulin in the blood sample of at least 5.6 ng/ml.

19. The method of treating a mammalian subject according to claim 16, wherein the blood product is plasma.


SEQUENCE LISTING (5 sequences)

SEQ ID NO: 1 - protein, 557 aa, unidentified
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 2 - protein, 497 aa, unidentified
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 3 - protein, 24 aa, unidentified
GLLIGISIASLCLVVALLALLCHL

SEQ ID NO: 4 - protein, 36 aa, unidentified
RKKQGAARAKMEYKCAAPSKEVVLQHVRTERTPQRL

SEQ ID NO: 5 - protein, 333 aa, unidentified
PAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECH